(19) United States
(12) Patent Application Publication
(10) Pub. No.: US 2010/0146671 A1
(43) Pub. Date: Jun. 10, 2010

(54) TARGETING VECTOR

(75) Inventors: Olivier Cotsaftis, South Australia (AU); Mark Alfred Tester, South Australia (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Australian Centre for Plant Functional Genomics Pty Ltd., Urrbrae, South Australia (AU)

(21) Appl. No.: 11/993,410

(22) PCT Filed: Jun. 23, 2006
(86) PCT No.: PCT/AU06/00875

§ 371 Date: Apr. 8, 2008

(30) Foreign Application Priority Data
Jun. 24, 2005 (AU) 2005903324

(51) Int. Cl. A01H 5/00; C12N 15/82; C12N 5/10; C12N 15/64

(52) U.S. Cl. 800/298; 435/320.1; 435/468; 435/419; 435/91.4

(57) ABSTRACT

The present invention relates generally to nucleic acid constructs, which are useful for inserting a nucleotide sequence of interest into a target nucleic acid molecule via homologous recombination. The present invention also relates to methods for producing such constructs.

### CROSS-REFERENCES TO RELATED APPLICATIONS

This application is a U.S. National Phase of PCT/AU2006/000875, filed Jun. 23, 2006, which claims the benefit of Australian Application No. 2005903324, filed Jun. 24, 2005, each of which are herein incorporated by reference for all purposes.

### FIELD OF THE INVENTION

The present invention relates generally to nucleic acid constructs, which are useful for inserting a nucleotide sequence of interest into a target nucleic acid molecule via homologous recombination. The present invention also relates to methods for producing such constructs.

### BACKGROUND OF THE INVENTION

Reference to any prior art in this specification is not, and should not be taken as, an acknowledgment or any form of suggestion that this prior art forms part of the common general knowledge in any country.

Despite the significant efforts being invested in the genome sequencing of higher plants, knowledge of gene function generally remains limited. For example, in rice (Oryza sativa), a highly accurate sequence of the japonica's cv. Nipponbare 12 chromosomes has been published by the International Rice Genome Sequencing Project (Nature, 436:793-800, 2005). However, of the 37,544 computer-predicted genes in the rice genome, roughly half of them have been assigned uncertain functions on the basis of their sequence, while only a number in the hundreds have been ascribed a precise and verified function.

In an attempt to assign a precise function to a given gene, large insertional mutant libraries have been developed in higher plants. Insertional mutagenesis, involving the random insertion of transposable elements or TDNA into the host genome to act as a molecular tag of the interrupted gene, has proven to be a valuable tool to address gene function through detailed analysis of mutant phenotypes. Systematic isolation and sequencing of genomic DNA flanking the insertion sites (known as FSTs or Flanking Sequence Tag) offers the opportunity to rapidly characterise plants altered in a candidate gene sequence. This approach is notably most useful in fully sequenced genomes such as in Arabidopsis thaliana wherein the sequences of approximately 296,000 FSTs have been published, representing a near saturation of the genome with insertion sites.

With 125 Mbp of nucleotide sequence encoding approximately 26,422 genes, the Arabidopsis genome shows very limited synteny with the 389 Mbp of sequence and 37,544 predicted genes of the rice genome. In contrast, almost all cereal genes are present in rice with a highly conserved macro-colinearity between genomes. For these reasons, there has been a need to specifically develop mutant libraries in rice. Chemically or physically mutagenized populations harbouring alterations from point mutations up to several kilobase deletions have been created and their use for PCR-based identification in DNA pools of a mutant line altered in a target candidate sequence has been undertaken. In parallel, several T-DNA, transposon and retrotransposon tagging populations have also been generated.

Despite their widespread use, libraries generated by chemical, physical or insertion mutagenesis also have drawbacks that can be common to all mutagens or specific to a given type. Since mutagenesis relies on the random creation of lesions throughout the genome followed by screening for a specific mutation, large collections of lines have to be generated and propagated in order to increase the chance of disrupting a particular gene. Considering that the number of lesions created in a single plant is variable, an observed mutant phenotype may not be correlated to a labelled tag or could be the result of several lesions. Backcrosses with a wild type parent are then often needed to remove additional lesions from the genetic background.

To bypass these limitations, the ability to target DNA integration at a given locus is of major interest to functional genomic projects. It would allow specific modification or disruption of endogenous genes, providing a tool for more detailed analysis of gene function. Such a technique would also permit the locus-specific integration of a transgene into a predetermined site of the host genome, avoiding the accidental inactivation of an endogenous gene localised at the insertion site or the unexpected expression profiles of the transgene itself. Furthermore, it is expected that sequencing alleles at particular loci of interest (both coding sequences and regulatory regions) among genetic resources of crop plants, as well as in establishing association between allele version and the agronomic value of a given trait is likely to become more prevalent. From this perspective, the development of a method which allows targeting of a particular gene would considerably enhance plant improvement efficiency.

Unfortunately, the natural propensity of DNA to recombine within homologous areas of the genome rarely happens in higher eukaryotes. Currently, this technique is applied routinely to organisms whose genomes preferentially use homologous recombination to mediate DNA integration, such as for example prokaryotes, yeast and the moss Physcomitrella patens. The cytoplasmic genomes of higher eukaryotes are also amenable to targeted modifications due to their prokaryotic origins. However, in the nuclear genomes of higher eukaryotes, DNA mostly integrates in a random manner, even when there are long stretches of homology shared within the genomic template.

Gene targeting has been described in higher plants, but typically only in dicotyledonous plants and generally with a very low frequency ranging from 10⁻³ to 10⁻⁶. However, an efficient method of T-DNA-mediated targeted disruption of a non-selectable locus (the waxy gene) has been reported in rice, which is predicated on the use of a large homologous region of 13 kb and highly expressed, duplicated diphtheria toxin gene-based counter-selectable markers (Terada et al., Nat Biotechnol, 20:1030-1034, 2002). The gene targeting events described in this paper occurred approximately once for every 99 escapes, resulting in an observable frequency of 1%.

However, gene-targeting constructs such as those used by Terada et al. (supra) are complex. These constructs are produced by the ligation of a range of DNA fragments and, as such, are labour intensive and relatively slow to construct. Furthermore, the non-modular nature of the construct means that a new targeting construct must be produced for each different nucleic acid target. Therefore, a more rapid an efficient method for assembling gene targeting constructs would be desirable.

### BRIEF SUMMARY OF THE INVENTION

The present invention is predicated, in part, on a targeting vector for inserting a nucleotide sequence of interest into a target nucleotide sequence in a cell via homologous recombination, as well as methods for generating such vectors. In a first aspect, the present invention contemplates a nucleic acid construct for inserting a nucleotide sequence of interest, nsoi, into a target nucleic acid molecule via homologous recombination, the nucleic acid construct comprising genetic element (I) as shown below:

[NSM1-RSα-5′HR-RSβ-nsoi-RSγ-3′HR-RSδ-NSM2] (I)

wherein:

NSM1 comprises a nucleotide sequence encoding a negative selectable marker, or optionally may be absent if a nucleotide sequence encoding a negative selectable marker is present at NSM2;

RSα comprises a nucleotide sequence defining a first recombination site;

5′HR comprises a nucleotide sequence which is homologous to a nucleotide sequence 5′ to, and/or inclusive of, a nucleotide sequence in the target nucleic acid molecule;

RSβ comprises a nucleotide sequence defining a second recombination site;

nsoi comprises a nucleotide sequence of interest to be inserted into the target nucleic acid molecule;

RSγ comprises a nucleotide sequence defining a third recombination site;

3′HR comprises a nucleotide sequence which is homologous to a nucleotide sequence 3′ to, and/or inclusive of, a nucleotide sequence in the target nucleic acid molecule;

RSδ comprises a nucleotide sequence defining a fourth recombination site;

NSM2 comprises a nucleotide sequence encoding a negative selectable marker, or optionally may be absent if a nucleotide sequence encoding a negative selectable marker is present at NSM1;

wherein homologous recombination between the regions of homology, 5′HR and/or 3′HR, and homologous nucleotide sequences in the target nucleic acid molecule results in insertion of the portion of genetic element (I) which is bounded by, and optionally inclusive of, 5′HR and 3′HR, into the target nucleic acid molecule.

In a preferred embodiment, the recombination sites, RSα, RSβ, RSγ and RSδ comprise lambda-phage att recombination sites. In one particularly preferred embodiment, RSα, RSβ, RSγ and RSδ comprise attB recombination sites.

The nucleic acid construct of the present invention may be used to insert a nucleotide sequence of interest into a nucleic acid molecule in a cell via homologous recombination.

Accordingly, in a second aspect, the present invention contemplates a method for inserting a nucleotide sequence of interest into a target nucleic acid molecule in a cell via homologous recombination, the method comprising introducing to the cell a nucleic acid construct comprising genetic element (I) as described according to the first aspect of the invention; and selecting transformants which comprise the inserted nucleotide sequence of interest on the basis of expression of a positive selectable marker incorporated into the nucleotide sequence of interest, if present, and/or the absence of expression of one or both of the negative selectable markers NSM1 and NSM2.

In third aspect, the present invention further extends to a cell comprising the nucleic acid construct according to the first aspect of the invention, or a genomically integrated form of the construct.

In a fourth aspect, the present invention should be understood to extend to a multicellular organism comprising one or more cells referred to in the third aspect of the invention.

In a fifth aspect, the present invention provides a method for producing a genetic construct comprising genetic element (I), the method comprising:

(i) providing one or more nucleic acid molecules which together comprise genetic elements (II), (III), (IV) and (V) as shown below:

[NSM1-RSa-RSd-NSM2] (II)

[RSa′-5′RH-RSb] (III)

[RSb′-nsoi-RSc′] (IV)

[RSc-3′RH-RSd′] (V)

wherein:

NSM1 comprises a nucleotide sequence encoding a negative selectable marker, or is optionally absent if a nucleotide sequence encoding a negative selectable marker is present at NSM2;

NSM2 comprises a nucleotide sequence encoding a negative selectable marker, or is optionally absent if a nucleotide sequence encoding a negative selectable marker is present at NSM1;

5′HR comprises a nucleotide sequence which is homologous to a nucleotide sequence 5′ to, and/or inclusive of, a nucleotide sequence in the target nucleic acid molecule;

nsoi comprises a nucleotide sequence of interest to be inserted into the target nucleotide sequence;

3′HR comprises a nucleotide sequence which is homologous to a nucleotide sequence 3′ to, and/or inclusive of, a nucleotide sequence in the target nucleic acid molecule; and

RSa is a nucleotide sequence defining a recombination site which can recombine with RSa′ when acted on by a recombinase; RSb is a nucleotide sequence defining a recombination site which can recombine with RSb′ when acted on by a recombinase, RSc is a nucleotide sequence defining a recombination site which can recombine with RSc′ when acted on by a recombinase and RSd is a nucleotide sequence defining a recombination site which can recombine with RSd′ when acted on by a recombinase; and

(ii) administering one or more recombinases to the one or more nucleic acid molecules comprising genetic elements (II), (III), (IV) and (V) such that RSa and RSa′ recombine to yield a recombination site RSα, RSb and RSb′ recombine to yield a recombination site RSβ, RSc and RSc′ recombine to yield a recombination site RSγ and RSd and RSd′ recombine to yield a recombination site RSδ, thereby generating a nucleic acid construct comprising genetic element (I).

In a sixth aspect, the present invention contemplates a genetic construct comprising genetic element (II) as defined in the fifth aspect of the invention.

In a seventh aspect, the present invention also provides a kit for performing the method of the fifth aspect of the invention, wherein the kit comprises at least the genetic construct of the sixth aspect of the invention.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

### BRIEF DESCRIPTION OF THE DRAWINGS

FIG. 1 shows a map of the vector pMET KO, which represents one preferred embodiment of the targeting vector of the present invention.

FIG. 2 shows a map of pMET KI, which represents another preferred embodiment of the targeting vector of the present invention.

FIG. 3 shows a map of the vector pMET, which is a preferred embodiment of the vector that can be used to generate the targeting vectors shown in FIGS. 1 and 2.

FIG. 4 shows a map of the modified pCAMBIA0380 plasmid. In this modified vector, the original kanamycin resistance gene for bacterial selection has been replaced with a spectinomycin resistance gene.

FIG. 5 shows a map of the pDESTR4-R3 plasmid.

FIG. 6 shows a map of the pDONRP4-P1R plasmid and the pENT/L4-R1R plasmid, the latter of which is produced as a result of lambda-phage recombinase mediated insertion of a targeting sequence (5′ region of homology) into the pDONRP4-P1R plasmid.

FIG. 7 shows a map of the pDONRP2R-P3 plasmid and the pENT/R2R-L3 plasmid, the latter of which is produced as a result of lambda-phage recombinase mediated insertion of a targeting sequence (3′ region of homology) into the pDONRP2R-P3 plasmid.

FIG. 8 shows a map of the pDONR221 plasmid.

FIG. 9 shows a map of pDONR221/Gal4::nptII::UAS, which is a modified pDONR221 plasmid carrying a Gal4::nptII::UAS knock in cassette.

FIG. 10 a map of pDONR221/PS4::hpt::T35S, which is a modified pDONR221 plasmid carrying a PS4::hpt::T35S knock out cassette.

### DESCRIPTION OF PREFERRED EMBODIMENTS

It is to be understood that the following description is for the purpose of describing particular embodiments only and is not intended to be limiting with respect to the above description.

DNA recombination is a tightly regulated process in which broken DNA ends, such as those left by double stranded breaks, are repaired. Furthermore, in some organisms, such as plants, rejoined double stranded breaks in a DNA molecule are also prone to DNA insertion at the site of the repair.

DNA recombination typically occurs via one of two different pathways homologous recombination (HR) or illegitimate recombination (IR) via non-homologous end joining (NHEJ).

As referred to herein, “homologous recombination” should be understood to refer to the recombination of a first DNA molecule with another DNA molecule with which the first DNA molecule has homology. A detailed description of homologous recombination mechanisms with particular reference to plant cells may be found in the review of Cotsaftis and Guiderdoni (Transgenic Research, 14:1-14, 2005).

Conversely, “illegitimate recombination” should be understood to refer to the joining of two broken DNA ends without a need for substantial homology (i.e., homology which is greater than micro-homology at the insertion sites) between the DNA ends.

The vector of the present invention may be used to insert a nucleotide sequence of interest into a target nucleic acid molecule in any cell type. As such, the term “cell” as used herein should be understood in its broadest context to include any cell type, including bacteria, archaea and eukaryotic cells including animal, plant and fungal cells.

Preferably, the cell is a eukaryotic cell. Even more preferably, the cell is a plant cell. Preferably, the term “plant cell” includes cells from, for example, monocotyledonous angiosperms (monocots), dicotyledonous angiosperms (dicots), gymnosperms and the like.

In a more preferred embodiment, the plant cell is derived from a monocotyledonous plant, more preferably a cereal crop plant.

As used herein, the term “cereal crop plant” may be a member of the Gramineae (grass family) that produces grain. Examples of Gramineae cereal crop plants include wheat, rice, maize, millets, sorghum, rye, triticale, oats, barley, teff, wild rice, spelt and the like. The term cereal crop plant should also be understood to include a number of non-Gramineae plant species that also produce edible grain, and are known as the pseudocereals, such as amaranth, buckwheat and quinoa.

Although cells derived from monocotyledonous plants such as cereal crop plants represent preferred cell types for use with the present invention, the present invention may also be used with dicotyledonous plant cells. Exemplary dicotyledonous plant cells that may be used in accordance with the present invention include, for example, cells derived Arabidopsis spp., Nicotiana spp., Brassica spp. and Glycine spp.

In a first aspect, the present invention contemplates a nucleic acid construct for inserting a nucleotide sequence of interest into a target nucleic acid molecule in a cell via homologous recombination, the nucleic acid construct comprising genetic element (I) as shown below:

[NSM1-RSα-5′HR-RSβ-nsoi-RSγ-3′HR-RSδ-NSM2] (I)

wherein:

NSM1 comprises a nucleotide sequence encoding a negative selectable marker, or optionally may be absent if a nucleotide sequence encoding a negative selectable marker is present at NSM2;

RSα comprises a nucleotide sequence defining a first recombination site;

5′HR comprises a nucleotide sequence which is homologous to a nucleotide sequence 5′ to, and/or inclusive of, a nucleotide sequence in the target nucleic acid molecule;

RSβ comprises a nucleotide sequence defining a second recombination site;

nsoi comprises a nucleotide sequence of interest to be inserted into the target nucleic acid molecule;

RSγ comprises a nucleotide sequence defining a third recombination site;

3′HR comprises a nucleotide sequence which is homologous to a nucleotide sequence 3′ to, and/or inclusive of, a nucleotide sequence in the target nucleic acid molecule;

RSδ comprises a nucleotide sequence defining a fourth recombination site;

NSM2 comprises a nucleotide sequence encoding a negative selectable marker, or optionally may be absent if a nucleotide sequence encoding a negative selectable marker is present at NSM1;

wherein homologous recombination between the regions of homology, 5′HR and/or 3′HR, and homologous nucleotide sequences in the target nucleic acid molecule results in insertion of the portion of genetic element (I) which is bounded by, and optionally inclusive of, 5′HR and 3′HR, into the target nucleic acid molecule.

The targeting vector of the present invention is useful for, inter alia, the site specific or targeted insertion of a nucleotide sequence of interest into a target nucleic acid molecule in a cell, via homologous recombination. Preferably, the “target nucleic acid molecule” may be any nucleic acid molecule that is present in a cell. More preferably, the target nucleic acid molecule may be a genomic DNA molecule, a chromosome, a mitochondrial DNA molecule, a plastid DNA molecule, a chloroplast DNA molecule, an artificial chromosome, a cosmid, a plasmid, and the like.

The site specificity of the insertion is conferred by the nucleotide sequences, 5′RH and 3′RH, in genetic element (I). As set out above, the nucleotide sequences designated 5′RH and 3′RH are homologous to a nucleotide sequence in and/or flanking the target nucleic acid molecule. These regions of homology facilitate homologous recombination between genetic element (I) and the target nucleic acid molecule.

Preferably, the nucleotide sequences of 5′RH and 3′RH are homologous with the target nucleic acid molecule over a nucleotide sequence length of at least 100 bp, more preferably at least 500 bp, and most preferably at least 5 kb. Preferably the 5′RH and 3′RH regions are at least 50% identical to the target nucleotide sequence, more preferably, the 5′RH and 3′RH regions are at least 75% identical to the target nucleotide sequence, even more preferably, the 5′RH and 3′RH regions are at least 90% identical to the target nucleotide sequence and most preferably the 5′RH and 3′RH regions are 95 to 100% identical to the target nucleotide sequence.

As set out above, the construct of the first aspect of the invention is suitable for insertion of a nucleotide sequence of interest, nsoi, into target nucleic acid molecule via homologous recombination. The inserted nucleotide sequence may be any sequence that is to be inserted into the target nucleotide sequence in the cell.

Preferably, the nucleotide sequence of interest comprises, inter alia, a nucleotide sequence that encodes a positive selectable marker. The nucleotide sequence encoding a “positive selectable marker” incorporated into the nucleotide sequence of interest allows the selection of transformants wherein the nucleotide sequence of interest has been inserted into the target nucleic acid molecule in the cell and is subsequently expressed by the cell. Nucleotide sequences which encode “positive selectable markers” include any nucleotide sequences which, when expressed by a cell, confer a phenotype on the cell that facilitates the identification and/or selection of these transformed cells. A range of nucleotide sequences encoding suitable positive selectable markers are known in the art. Exemplary nucleotide sequences that encode positive selectable markers include: antibiotic resistance genes such as ampicillin-resistance genes, tetracycline-resistance genes, kanamycin-resistance genes, the AURI-C gene which confers resistance to the antibiotic aureobasidin A, neomycin phosphotransferase genes (e.g., nptI and nptII) and hygromycin phosphotransferase genes (e.g., hpt); herbicide resistance genes including glufosinate, phosphinothricin or bialaphos resistance genes such as phosphinothricin acetyl transferase encoding genes (e.g., bar), glyphosate resistance genes including 3-enoyl pyruvyl shikimate 5-phosphate synthase encoding genes (e.g., aroA), bromyxnil resistance genes including bromyxnil nitrilase encoding genes, sulfonamide resistance genes including dihydropterate synthase encoding genes (e.g., sul) and sulfonylurea resistance genes including acetolactate synthase encoding genes; enzyme-encoding reporter genes such as GUS and chloramphenicolacetyltransferase (CAT) encoding genes; fluorescent reporter genes such as the green fluorescent protein-encoding gene; and luminescence-based reporter genes such as the luciferase gene, amongst others.

In one preferred embodiment, the nucleotide sequence encoding a positive selectable marker comprises an antibiotic resistance gene. In more preferred embodiments, the antibiotic resistance gene comprises a neomycin phosphotrasferase gene (e.g., nptI and/or nptII) and/or a hygromycin phosphotransferase gene (e.g., hpt).

In another preferred embodiment, the nucleotide sequence encoding a positive selectable marker is a herbicide resistance gene as described above.

The nucleotide sequence of interest may also comprise one or more control sequences. The term “control sequences” should be understood to include all components known in the art, which are necessary or advantageous for the transcription, translation and or post-translational modification of an operably connected nucleotide sequence, or the transcript or protein encoded thereby. Each control sequence may be native or foreign to the nucleotide sequence. The control sequences may include, but are not limited to, a leader, polyadenylation sequence, propeptide sequence, promoter, enhancer or upstream activating sequence, signal peptide sequence, and transcription terminator. Typically, a control sequence at least includes a promoter.

The term “promoter” as used herein, describes any nucleic acid that confers, activates or enhances expression of a nucleic acid molecule in a cell. Promoters are generally positioned 5′ (upstream) to the nucleotide sequences that they control. In the construction of heterologous promoter/structural gene combinations, it is generally preferred to position the promoter at a distance from the transcription start site that is approximately the same as the distance between that promoter and the gene it controls in its natural setting, ie. the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, ie. the genes from which it is derived. Again, as is known in the art, some variation in this distance can also occur.

A promoter may regulate the expression of an operably connected nucleotide sequence constitutively, or differentially with respect to the cell, tissue, organ or developmental stage at which expression occurs, or in response to external stimuli such as physiological stresses, or pathogens, or metal ions, amongst others. As such, the present invention contemplates the use of any promoter that is active in a cell of interest. A wide array of promoters, which are active in any of bacteria, fungi, animal cells or plant cells are known in the art.

In preferred embodiments of the invention, wherein plant cells are used, plant-active constitutive, inducible, tissue-specific or activatable promoters are particularly preferred.

Constitutive promoters typically direct expression in nearly all cell types or tissues of a particular organism and are largely independent of environmental and developmental factors. Examples of plant-active constitutive promoters that may be used in accordance with preferred embodiments of the present invention include plant viral derived promoters such as the Cauliflower Mosaic Virus 35S and 19S (CaMV 35S and CaMV 19S) promoters; bacterial plant pathogen derived promoters such as opine promoters derived from Agrobacterium spp., e.g., the Agrobacterium-derived nopaline synthase (nos) promoter; and plant-derived promoters such as the rubisco small subunit gene (rbcS) promoter, the plant ubiquitin promoter (Pubi) and the rice actin promoter (Pact).

“Inducible” promoters are promoters that may be induced by the presence or absence of biotic or abiotic factors. The term “inducible promoters” should be understood to include chemically inducible promoters and physically inducible promoters. Exemplary plant-active chemically inducible promoters that may be used accordance with preferred embodiments of the present invention include promoters that have activity which is regulated by chemical compounds such as alcohols, antibiotics, steroids, metal ions or other compounds. Examples of chemically inducible promoters include: alcohol-regulated promoters (e.g., see European Patent 637 339 and U.S. Pat. No. 6,605,754); tetracycline-regulated promoters (e.g., see U.S. Pat. No. 5,851,796 and U.S. Pat. No. 5,464,758); steroid-responsive promoters such as glucocorticoid receptor promoters (e.g., see U.S. Pat. No. 5,512,483), estrogen receptor promoters (e.g., see European Patent Application 1 232 273), ecdysone receptor promoters (e.g., see U.S. Pat. No. 6,379,945) and the like; metal-responsive promoters such as metallothionein promoters (e.g., see U.S. Pat. No. 4,940,661, U.S. Pat. No. 4,579,821 and U.S. Pat. No. 4,601,978); and pathogenesis related promoters such as chitinase or lysozyme promoters (e.g., see U.S. Pat. No. 5,654,414) or PR protein promoters (e.g., see U.S. Pat. No. 5,689,044, U.S. Pat. No. 5,789,214, Australian Patent 708850, U.S. Pat. No. 6,429,362).

The inducible promoter may also be a physically regulated promoter, which is regulated by non-chemical environmental factors such as temperature (both heat and cold), light and the like. Examples of plant-active physically regulated promoters include heat shock promoters (e.g., see U.S. Pat. No. 5,447,858, Australian Patent 732872, Canadian Patent Application 1324097); cold inducible promoters (e.g., see U.S. Pat. No. 6,479,260, U.S. Pat. No. 6,084,08, U.S. Pat. No. 6,184,443 and U.S. Pat. No. 5,847,102); light inducible promoters (e.g., see U.S. Pat. No. 5,750,385 and Canadian Patent 132 1563); light repressible promoters (e.g., see New Zealand Patent 508103 and U.S. Pat. No. 5,639,952).

Tissue specific promoters include promoters that are preferentially or specifically expressed in one or more specific tissue types and/or one or more developmental stages of a multicellular organism. As such tissue specific promoters may be used to specifically activate the expression of an operably connected nucleotide sequence in one or more specific tissues and/or at one or more specific developmental stages of a multicellular organism. It should be understood that a tissue specific promoter may be either constitutive or inducible. Examples of plant-active tissue specific promoters that may be used in accordance with the present invention include: root specific promoters such as those described in U.S. Patent Application 2001047525; fruit specific promoters including ovary specific and receptacle tissue specific promoters such as those described in European Patent 316 441, U.S. Pat. No. 5,753,475 and European Patent Application 973 922; and seed specific promoters such as those described in Australian Patent 612326 and European Patent application 0 781 849 and Australian Patent 746032.

The promoter may also be a promoter that is activatable by one or more transcriptional activators, referred to herein as an “activatable promoter”. For example, the activatable promoter may comprise a minimal promoter operably connected to an Upstream Activating Sequence (UAS), which comprises, inter alia, a DNA binding site for one or more transcriptional activators.

As referred to herein the term “minimal promoter” should be understood to include any promoter that incorporates at least a TATA box and transcription initiation site and optionally one or more CAAT boxes. More preferably, when the cell is a plant cell, the minimal promoter may be derived from the Cauliflower Mosaic Virus 35S (CaMV 35S) promoter. Preferably, the CaMV 35S derived minimal promoter may comprise a sequence that corresponds to positions −90 to +1 (the transcription initiation site) of the CaMV 35S promoter (also referred to as a −90 CaMV 35S minimal promoter), −60 to +1 of the CaMV 35S promoter (also referred to as a −60 CaMV 35S minimal promoter) or −45 to +1 of the CaMV 35S promoter (also referred to as a −45 CaMV 35S minimal promoter).

As set out above, the activatable promoter may comprise a minimal promoter fused to an Upstream Activating Sequence (UAS). The UAS may be any sequence that can bind a transcriptional activator to activate the minimal promoter. Exemplary transcriptional activators include, for example: yeast derived transcription activators such as Gal4, Pdr1, Gcn4 and Ace1; the viral derived transcription activator, VP16; Hap1 (Hach et al., J Biol Chem, 278:248-254, 2000); Gaf1 (Hoe et al., Gene, 215(2):319-328, 1998); E2F (Albani et al., J Biol Chem, 275:19258-19267, 2000); HAND2 (Dai and Cserjesi, J Biol Chem, 277:12604-12612, 2002); NRF-1 and EWG (Herzig et al., J Cell Sci, 113:4263-4273, 2000); P/CAF (Itoh et al., Nucl Acids Res, 28:4291-4298, 2000); MafA (Kataoka et al., J Biol Chem, 277:49903-49910, 2002); human activating transcription factor 4 (Liang and Hai, J Biol Chem, 272:24088-24095, 1997); Bcl10 (Liu et al., Biochem Biophys Res Comm, 320(1):1-6, 2004); CREB-H (Omori et al., Nucl Acids Res, 29:2154-2162, 2001); ARR1 and ARR2 (Sakai et al., Plant J, 24(6):703-711, 2000); Fos (Szuts and Bienz, Proc Natl Acad Sci USA, 97:5351-5356, 2000); HSF4 (Tanabe et al., J Biol Chem, 274:27845-27856, 1999); MAML1 (Wu et al., Nat Genet, 26:484-489, 2000).

In one preferred embodiment, the UAS comprises a nucleotide sequence that is able to bind to at least the DNA-binding domain of the GAL4 transcriptional activator. UAS sequences, which can bind transcriptional activators that comprise at least the GAL4 DNA binding domain, are referred to herein as UAS_{G}. In a particularly preferred embodiment, the UAS_{G} comprises the sequence 5′-CGGAGTACTGTCCTCCGAG-3′ or a functional homolog thereof.

As referred to herein, a “functional homolog” of the UAS_{G} sequence should be understood to refer to any nucleotide sequence which can bind at least the GAL4 DNA binding domain and which preferably comprises a nucleotide sequence having at least 50% identity, more preferably at least 65% identity, even more preferably at least 80% identity and most preferably at least 90% identity with the UAS_{G} nucleotide sequence.

The UAS sequence in the activatable promoter may comprise a plurality of tandem repeats of a DNA binding domain target sequence. For example, in its native state, UAS_{G} comprises four tandem repeats of the DNA binding domain target sequence. As such, the term “plurality” as used herein with regard to the number of tandem repeats of a DNA binding domain target sequence should be understood to include at least 2 tandem repeats, more preferably at least 3 tandem repeats and even more preferably at least 4 tandem repeats.

As mentioned above, the control sequences may also include a terminator. The term “terminator” refers to a DNA sequence at the end of a transcriptional unit which signals termination of transcription. Terminators are 3′-non-translated DNA sequences generally containing a polyadenylation signal, which facilitates the addition of polyadenylate sequences to the 3′-end of a primary transcript. As with promoter sequences, the terminator may be any terminator sequence that is operable in the cells, tissues or organs in which it is intended to be used. Examples of suitable terminator sequences which may be useful in plant cells include: the nopaline synthase (nos) terminator, the CaMV 35S terminator, the octopine synthase (ocs) terminator, potato proteinase inhibitor gene (pin) terminators, such as the pinII and pinIII terminators and the like.

In one preferred embodiment, the nucleotide sequence of interest in genetic element (I) may be a “knock out” cassette. As referred to herein, a knock out cassette refers to any nucleotide sequence which may be introduced into a target nucleotide sequence (e.g., a target gene) to disrupt, inhibit or reduce the transcription and/or translation of the target nucleotide sequence. Preferably, the knock out cassette comprises a positive selectable marker operably connected to a promoter and terminator.

A knock out cassette may inactivate a target gene or other expressed nucleic acid sequence via insertional mutagenesis, wherein a disrupting nucleotide sequence (such as a positive selectable marker) is inserted into a target nucleic acid in a cell. Insertion of the disrupting sequence inhibits, reduces or eliminates the transcription and/or translation of the gene, thereby reducing or eliminating the expression of the target nucleotide sequence in the cell.

In another preferred embodiment, the nucleotide sequence of interest may also comprise a “knock in” cassette. Preferably, the knock in cassette comprises a promoter which is positioned within the cassette such that the promoter can effect transcriptional control of a gene which is downstream (3′) of the insertion site of the targeting vector, when the targeting vector is inserted into a target nucleic acid. As such, a targeting vector carrying a knock in cassette may be used bring the expression of a gene or other nucleotide sequence of interest in a cell under the control of a promoter in the knock in cassette. The choice of promoter used in the knock in cassette depends on the desired level of expression of the subject gene or other nucleotide sequence. Thus, promoters with increased or decreased expression (relative to a native promoter of a target gene) may be used to effect increased or decreased levels of expression. Alternatively, promoters having altered spatial, temporal or incibility characteristics (again relative to a native promoter of a targeted gene) may be used to alter the spatial pattern, temporal pattern or incibility of expression of a target gene.

In a more preferred embodiment, the promoter in the “knock in” cassette may comprise an activatable promoter, as defined herein, which effects a knock in as described above, wherein the expression of the gene to be targeted is placed under the control of a transcription activator, such as GAL4.

The knock in cassette may also be adapted to provide both activatable and inducible expression of a target nucleic acid. For example, the second promoter may be operably connected to a second transcription activator, which in conjunction with an inducer, may activate a third promoter in the knock in cassette. The third promoter may then be used to control the expression of a nucleotide sequence downstream of the insertion site of the knock in cassette, thereby establishing the need for both the transcription activator (e.g., GAL4) and the inducer of the third promoter to drive expression of the target nucleotide sequence. An exemplary inducible expression system that may be used in conjunction with an activatable promoter to establish both activatable and inducible expression is the alcohol regulated expression system described in U.S. Pat. No. 6,605,754 and European Patent 637 339.

The knock in cassette may also comprise an appropriately positioned positive selectable marker to enable selection of transformants carrying the cassette.

In another embodiment, the nucleotide sequence of interest may also include any gene of interest to be expressed in a target cell. The gene may be operably connected to a promoter and/or terminator or may be adapted to be expressed from an endogenous promoter in the target nucleic acid. Furthermore, if the gene of interest gives a screenable phenotype, the nucleotide sequence of interest need not include an additional positive selectable marker. In one preferred embodiment, the nucleotide sequence of interest may be a replacement allele which comprises one or more nucleotide insertions, deletions or substitutions relative to a nucleotide sequence in the target nucleic acid molecule.

As set out above, the targeting vector of the present invention also comprises nucleotide sequences that encode one or more negative selectable markers, i.e., NSM1 and NSM2. Each of NSM1 and NSM2 comprises a nucleotide sequence encoding a negative selectable marker, or is optionally absent if a nucleotide sequence encoding a negative selectable marker is present at the other negative selectable marker. As such, genetic element (I) may comprise a nucleotide encoding a negative selectable marker at both NSM1 and NSM2, or may encode a selectable marker at only one of NSM1 or NSM2. In a preferred embodiment, both NSM1 and NSM2 in genetic element (I) comprise a nucleotide sequence encoding a negative selectable marker.

As used herein, the term “negative selectable marker” refers to a nucleotide sequence which, when expressed, inhibits the growth of a cell expressing the negative selectable marker in a particular environment and/or kills a cell which expresses the negative selectable marker.

When homologous recombination occurs between the 5′RH/3′RH sequences in genetic element (I) and homologous sequences in the target nucleic acid, the nucleic acid region between 5′RH and 3′RH in genetic element (I) is inserted into the target nucleic acid, while those regions outside the 5′RH and 3′RH regions are excluded. Therefore, when homologous recombination occurs between genetic element (I) and the target nucleic acid molecule, the negative selectable marker or markers are not incorporated into the target nucleic acid in the cell. Therefore, when homologous recombination occurs, the negative selection marker or markers are not expressed and therefore homologous recombinants may be selected on the basis of an absence of expression of the negative selectable marker or markers.

In contrast, random or illegitimate insertion of genetic element (I) into the target nucleic acid molecule may lead to insertion of one or both of the negative selectable markers together with the nucleotide sequence of interest into the target nucleic acid molecule. Therefore, in the situation of random or illegitimate insertion, the negative selectable marker or markers would be expressed, which would lead to inhibition of the growth of the cell expressing the negative selectable marker in a particular environment, and/or death of the cell which expresses the negative selectable marker.

In one preferred embodiment, one or both of NSM1 and NSM2 comprise a nucleotide sequence encoding a diphtheria toxin, an active fragment thereof or a functional homolog of the diphtheria toxin or active fragment thereof.

The diphtheria toxin is encoded by a phage that infects Corynebacterium diphtheriae. The diphtheria toxin itself is a two component bacterial exotoxin synthesized as a single polypeptide chain containing an A (active) domain and a B (binding) domain. Fragment A of the toxin is responsible for enzymatic ADP-ribosylation of elongation factor 2, while fragment B is responsible for binding of toxin to cell receptors and entry of fragment A.

After cleavage and release from fragment B, the A fragment is able to catalyze the covalent attachment of the ADP ribose moiety of NAD to elongation factor 2. The transfer of ADP-ribose from NAD to the eukaryotic elongation factor 2 inhibits the function of elongation factor 2 in protein synthesis. Ultimately, inactivation of all of the host cell EF-2 molecules causes death of the cell.

An exemplary amino acid sequence defining a diphtheria toxin is set out in Entrez Accession No. P00588. Furthermore, as used herein, a “functional homolog” of the diphtheria toxin should be understood to include any protein that has substantially similar activity to the diphtheria toxin and which preferably comprises at least 50% amino acid sequence identity to Entrez Accession No. P00588. More preferably, the functional homolog comprises at least 65% sequence identity, yet more preferably at least 80% sequence identity, even more preferably at least 90% sequence identity, and even more preferably at least 95% sequence identity and most preferably at least 100% sequence identity to Entrez Accession No. P00588. When determining the amino acid sequence identity the amino acid sequences should be compared to the diphtheria toxin reference sequence over a comparison window of at least 100 amino acid residues, more preferably at least 200 amino acid residues, yet more preferably at least 300 amino acid residues, even more preferably at least 400 amino acid residues and most preferably over the full length of Entrez Accession No. P00588.

The negative selection markers, NSM1 and NSM2 may also encode an active fragment of the diphtheria toxin or a functional homolog thereof. For example, as set out above, the intact diphtheria toxin molecule is inactive until proteolytic changes separate the inactive precursor into fragments A and B. Fragment A has enzymatic activity, while at least in human cells, fragment B attaches to cellular receptors and grants fragment A entrance into the cell where it inhibits protein synthesis.

In a preferred embodiment, one or both NSM1 and NSM2 each encode a nucleotide sequence encoding at least an enzymatically active fragment or subunit of the diphtheria toxin or a functional homolog thereof. More preferably, NSM1 and NSM2 comprise a nucleotide sequence encoding at least an A fragment of the diphtheria toxin or a functional homolog thereof. An exemplary amino acid sequence of the A fragment of the diphtheria toxin is set out in residues 33 to 225 of Entrez Accession No. P00588. Most preferably, NSM1 and NSM2 comprise a nucleotide sequence defining a DT-A gene. As used herein, “a DT-A gene” should be understood to encompass any nucleic acid sequence that encodes at least the A fragment of the diphtheria toxin or a functional homolog thereof.

Although nucleic acid molecules which encode the diphtheria toxin, the diphtheria toxin A subunit, or functional homologs thereof represent preferred negative selection markers, the present invention should not be considered limited to only those markers. For example, a range of other negative selection markers are known in the art and these should be considered within the scope of the invention. Exemplary negative selection markers which may also be used in accordance with the present invention include: the tms2 gene (Upadhyaya et al., Plant Mol. Biol. Rep., 18:227-233, 2000); the bacterial codA gene and bacterial p450 monooxygenase as described by Koprek et al. (Plant J., 19(6):719-726, 1999); antisense sequences to the positive selection marker used in the nucleotide sequence of interest, e.g., the anti-npt genes described by Xiang and Guerra (Plant Physiol., 102(1):287-293); and the like.

Furthermore, each of NSM1 and NSM2 may encode the same or different negative selectable markers.

In order for the negative selectable markers to be expressed in a cell, they are preferably connected to control sequences, more preferably at least a promoter, as hereinbefore defined.

Preferably, the control sequences for each of NSM1 and NSM2 are substantially only active the cell containing the target nucleic acid molecule, and not in any other cell type. More preferably, the negative selectable markers are not expressed in any cell that is used in the construction or delivery of genetic element (I) to the target cell. Expression of the negative selectable markers cells used in the construction and/or delivery of genetic element (I) may effect selection against these cells, thereby substantially reducing the cloning efficiency and reducing the ability to maintain a nucleic acid molecule comprising the DT-A expression cassette in a cell.

In a more preferred embodiment, wherein the target nucleic acid molecule is a nucleic acid molecule in a plant cell, the promoters controlling the expression of NSM1 and/or NSM2 are preferably not active in bacterial cells and more preferably only substantially active in plant cells. Exemplary promoters which exhibit substantial activity in plant cells and substantially no activity in bacterial cells include, for example, the plant ubiquitin promoter (Pubi) and the rice actin promoter (Pact).

In one preferred embodiment, the plant-active promoter comprises a promoter other than the CaMV 35S promoter.

The control sequences of NSM1 and NSM2 may be the same or different, although in a more preferred embodiment, the control sequences for NSM1 and NSM2, particularly the promoters, are different.

The negative selectable markers may be incorporated into genetic element (I) in any orientation, for example, a nucleotide sequences encoding a negative selectable marker may be transcribed from a flanking region of genetic element (I) in towards the nucleotide sequence of interest. Alternatively, a nucleotide sequence encoding a negative selectable marker may be transcribed from an end proximal to the nucleotide sequence of interest out toward the flanking regions of genetic element (I).

In one preferred embodiment, the nucleotide sequences encoding the negative selectable markers are both transcribed from a flanking region of genetic element (I) in towards the nucleotide sequence of interest. In this arrangement, if genetic element (I) is transferred into a plant cell as a T-DNA from Agrobacterium and the T-DNA is aberrantly truncated when entering the nucleus, one or both of the promoters controlling the negative selectable markers may become truncated. However, the negative selectable marker genes per se may still remain intact. Therefore, these genes may still be expressed from an endogenous promoter in the target nucleic acid.

As set out above, the construct of the first aspect of the invention further includes nucleotide sequences defining four recombination sites, RSα, RSβ, RSγ and RSδ. These recombination sites facilitate the site-specific insertion or excision of sequences which are bounded by the recombination sites. As such, the recombination sites allow the insertion or excision of nucleotide sequences into genetic element (I) either before or after insertion of the nucleotide sequence of interest into the target nucleotide sequence in the cell. Furthermore, the recombination sites in genetic element (I) may also be the product of assembling the targeting vector according to a preferred method of assembly, as described hereafter in later aspects of the invention.

The present invention contemplates any suitable recombination sites known in the art and, therefore, the present invention should not be considered limited by the specific nucleotide sequence of the recombination site.

However, in a preferred embodiment, the recombination sites, RSα, RSβ, RSγ and RSδ comprise lambda-phage att recombination sites.

Lambda phage recombination occurs following two pairs of strand exchanges and ligation of the DNA. Lambda recombination through att sites is catalysed by a mixture of enzymes that (i) bind to the att sites; (ii) bring the interacting att sites together; (iii) cleave the att sites; and (iv) ligate the DNA molecules to yield the recombination products. The proteins involved differ depending on the particular att sites which are to be recombined.

Typically, attB sites recombine with attP sites and attL sites recombine with attR sites. Furthermore, the recombination of an attB with an attP site yields an attL and an attR site, similarly, the recombination of an attL with an attR site yields an attB and an attP site.

The recombination of attB with attP is catalysed by the lambda lysogenic pathway, which is catalyzed by the bacteriophage lambda integrase (Int) and the E. Coli Integration Host Factor (IHF) as described by Hartley et al. (Genome Res, 10:1788-1795, 2000). These enzymes would be readily obtained by one of skill in the art, for example, they are available in a pre-mixed form as BP Clonase enzyme mix from Invitrogen.

The recombination of attL with attR is catalysed by the lambda lytic pathway, which is catalyzed by the bacteriophage lambda integrase (Int) and excisionase (Xis) and the E. coli Integration Host Factor (IHF) as described by Hartley et al. (supra). Again, these enzymes would be readily obtained by one of skill in the art, and are, for example, available in a pre-mixed form as LR Clonase enzyme mix from Invitrogen.

Therefore, in a more preferred embodiment, RSα, RSβ, RSγ and RSδ in the targeting construct of the first aspect of the invention, comprise lambda phage att sites. Preferably, each of RSα, RSβ, RSγ and RSδ are each of the same class of att site (i.e., attB, attP, attL or attR), although they need not have the same nucleotide sequence as several variants within each class of att site are known.

The attL, attR, attB and/or attP recombination site sequences contemplated by the present invention may be either the wild type attL, attR, attB and/or attP recombination site sequences or may be derivatives thereof. Exemplary derivatives of the wild type attL, attR, attB and/or attP recombination site sequences that are particularly useful in accordance with the present invention include the modified attL, attR, attB and/or attP recombination site sequences developed by Clontech (e.g., see Clontech Gateway Technology brochure, Version E, dated 22 Sep. 2003).

In a more preferred embodiment, RSα, RSβ, RSγ and RSδ comprise attB recombination sites.

In a further preferred embodiment, the construct of the first aspect of the invention is adapted to be transferred into a plant via Agrobacterium-mediated transformation. Accordingly, in a particularly preferred embodiment, the construct according to the first aspect of the present is flanked by a left and/or right T-DNA border sequence(s). Suitable T-DNA border sequences would be readily ascertained by one of skill in the art. However, the term “T-DNA border sequences” should be understood to encompass any substantially homologous and substantially directly repeated nucleotide sequences that delimit a nucleic acid molecule that is transferred from an Agrobacterium sp. cell (or other plant transformation competent bacteria) into a plant cell susceptible to bacterial-mediated transformation. By way of example, reference is made to Peralta and Ream (Proc. Natl. Acad. Sci. USA, 82(15):5112-5116, 1985) and Gelvin (Microbiology and Molecular Biology Reviews, 67(1):16-37, 2003).

Although in one preferred embodiment, the construct of the first aspect of the invention is adapted to be transferred into a plant via Agrobacterium-mediated transformation, the present invention also contemplates any suitable modifications to the genetic construct which facilitate bacterial mediated insertion into a plant cell via bacteria other than Agrobacterium sp., as described in Broothaerts et al. (Nature, 433:629-633, 2005).

In addition to genetic element (I) as hereinbefore described, the genetic construct of the first aspect of the present invention may further comprise a range of other elements that facilitate the construction, cloning and/or selection for the construct in one or more different cell types. For example, the construct may comprise: a further selectable marker gene which is active in one or more hosts; any sequences which enable the transfer of the construct from one cell to another; and/or an origin of replication which is active in one or more cell types.

Accordingly, the present invention extends to all nucleic acid constructs essentially as described herein, which may include further nucleotide sequences intended for the maintenance and/or replication of the nucleic acid construct in prokaryotes or eukaryotes and/or the integration of said genetic construct or a part thereof into the genome of a eukaryotic or prokaryotic cell.

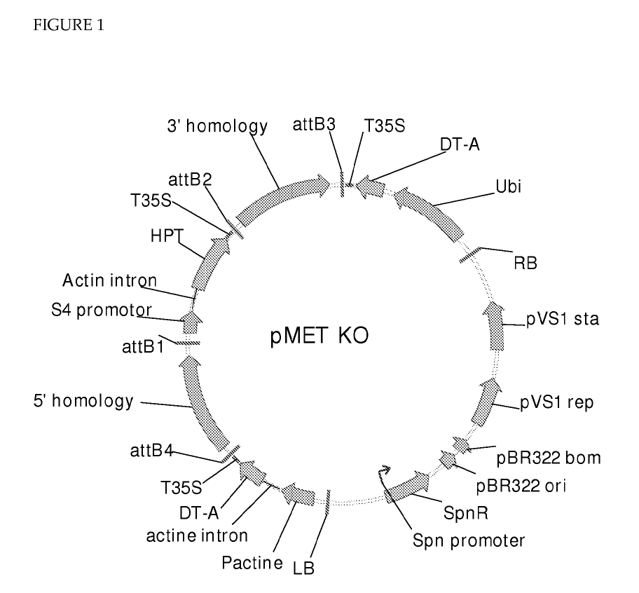

In one particularly preferred embodiment, the genetic construct according to the first aspect of the invention comprises the pMET KO vector illustrated in FIG. 1.

With reference to FIG. 1, the pMET KO vector comprises a preferred embodiment of genetic element (I). Specifically, in pMET KO, NSM1 comprises a DT-A gene under the control of the rice actin promoter (Pact) and the CaMV 35S terminator (T35S), which is shown immediately inside the T-DNA left border (LB) in pMET KO as shown in FIG. 1. NSM2 comprises a DT-A gene under the control of the ubiquitin promoter (Pubi) and a second T35S terminator, which is shown immediately inside the T-DNA right border (RB) in pMET KO.

In pMET KO, the nucleotide sequence of interest, nsoi, includes a positive selectable marker including a hygromycin phosphotransferase gene (hpt), together with an actin intron and CAT intron, under the control of the PS4 promoter (PS4) and CaMV 35S terminator (T35S). When expressed, the hpt gene confers resistance to hygromycin on a cell.

The regions of homology, 5′HR and 3′HR, which effect homologous recombination between pMET KO and a target nucleotide sequence, are shown in FIG. 1 as “5′ homology” and “3′ homology”. The recombination sites, RSα, RSβ, RSγ and RSδ are represented in FIG. 1 by the attB4, attB1, attB2 and attB3 sites, respectively.

pMET KO may be used to, among other things, effect site-directed insertional mutagenesis (i.e., knock out) of a gene of interest in a plant cell.

For example, the region of pMET KO bounded by the T-DNA border sequences, LB and RB in FIG. 1, may be introduced into a plant cell via Agrobacterium-mediated transformation. Once in the cell, the hpt positive selectable marker may insert into a target gene via homologous recombination between the regions of homology shared between pMET KO and the nucleotide sequence of the target gene. When insertion occurs via homologous recombination, the nucleotide sequence intermediate the regions of homology, optionally together with all or part of the regions of homology per se, is inserted into the target nucleic acid and the DT-A negative selectable markers are not inserted and therefore not expressed by the cell. Accordingly, when homologous recombination occurs, the target gene is disrupted by insertion of the hpt positive selectable marker and homologous recombinants may be selected on the basis of hygromycin resistance.

Alternatively, if illegitimate recombination occurs, the gene of interest has not been targeted and all of the Agrobacterium-introduced nucleic acid molecule may be inserted into the genome of the cell, including the DT-A negative selectable markers. In this case, although the hpt positive selectable marker may be expressed conferring hygromycin resistance on the cell, one or both of the DT-A negative selectable markers are also expressed. Expression of the DT-A gene causes the production of diphtheria toxin in the cell and, therefore, provides selection against transformants in which illegitimate recombination has occurred. Selection for the expression of the hpt positive selectable marker together with the negative selection provided by expression of the DT-A negative selectable markers, allows selection of transformants in which homologous recombination has occurred and, therefore, selection of recombinants in which a gene of interest has been targeted.

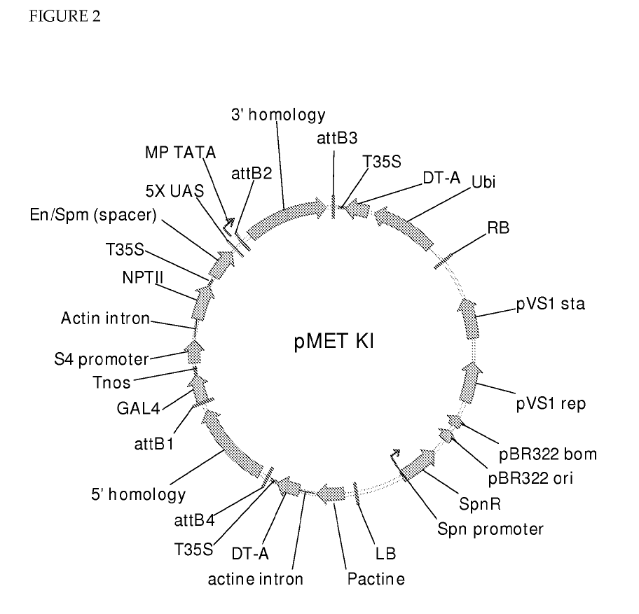

In another particularly preferred embodiment, the genetic construct according to the first aspect of the invention comprises pMET KI as shown in FIG. 2.

As can be seen, this vector is similar to pMET KO, other than it carries a gal4::nptII::UAS knock in cassette instead of the PS4::hpt::T35S knock out cassette as in pMET KO.

As shown in FIG. 2, the knock in cassette of pMET KI comprises (from 5′ to 3′) the GAL4 transcriptional activator coding region terminated by a nopaline synthase terminator. Downstream of this is an nptII positive selectable marker gene operably connected to an S4 promoter/actin intron and T35S terminator. Further downstream in the cassette is a five-time tandem repeat of the GAL4 binding site (UAS) which is operably connected to a minimal promoter (MP TATA).

In pMET KI the GAL4-UAS transactivation system is targeted in between the promoter and the ORF of the targeted gene. Thus, GAL4 in the knock in cassette is placed under the transcriptional control of the native promoter of the targeted gene. The GAL4 expressed from this sequence binds to the UAS element in the knock in cassette, which, in turn, drives the expression of the targeted gene. This system allows the over-expression of the targeted gene, while retaining the spatial and temporal specificity of the native promoter of the targeted gene.

In between the GAL4 gene and the UAS element, the nptII gene allows the selection of the recombinant lines while the spacer composed of a ˜0.9 kb fragment of the En/Spm transposon helps to suppress potential expression of the targeted gene by the S4 promoter driving the nptII selection gene.

As set out above, the nucleic acid construct of the present invention may be used to insert a nucleotide sequence of interest into a nucleic acid molecule in a cell via homologous recombination.

Accordingly, in a second aspect, the present invention contemplates a method for inserting a nucleotide sequence of interest into a nucleic acid molecule in a cell via homologous recombination, the method comprising administering to the cell a nucleic acid construct comprising genetic element (I) as described according to the first aspect of the invention; and selecting transformants which comprise the inserted nucleotide sequence of interest on the basis of the presence and/or expression of the nucleotide sequence of interest in the cell, and/or the absence of expression of one or both of the negative selectable markers NSM1 and NSM2 in the cell.

Methods of “administration” of a nucleic acid construct to a range of different cell types are known in the art. Preferably, the nucleic acid construct is administered to a cell via transformation. Suitable transformation protocols for the particular cell of interest may be readily ascertained by one of skill in the art. In preferred embodiments of the invention, wherein the cell is a plant cell, any method known in the art that is appropriate for the particular plant species may be used. Common methods include Agrobacterium-mediated transformation, microprojectile bombardment based transformation methods and direct DNA uptake based methods. Roa-Rodriguez et al. (Agrobacterium-mediated transformation of plants, 3^{rd} ed., CAMBIA Intellectual Property Resource, Canberra, Australia, 2003) review a wide array of suitable Agrobacterium-mediated plant transformation methods for a wide range of plant species. One preferable Agrobacterium-mediated transformation method which is particularly applicable to the transformation of rice is described in Sallaud et al., Theor. Appl. Genet., 106:1396-1408, 2003. Plant transformation using bacteria other than Agrobacterium spp. are also described in Broothaerts et al. (2005, supra). Microprojectile bombardment may also be used to transform plant tissue and methods for the transformation of plants, particularly cereal plants, and such methods are reviewed by Casas et al. (Plant Breeding Rev., 13:235-264, 1995). Direct DNA uptake transformation protocols such as protoplast transformation and electroporation are described in detail in Galbraith et al. (eds.) (Methods in Cell Biology, Vol. 50, Academic Press, San Diego, 1995). In addition to the methods mentioned above, a range of other transformation protocols may also be used. These include infiltration, electroporation of cells and tissues, electroporation of embryos, microinjection, pollen-tube pathway, silicon carbide and liposome-mediated transformation. Methods such as these are reviewed generally by Rakoczy-Trojanowska (Cell. Mol. Biol. Lett., 7:849-858, 2002). A range of other plant transformation methods may also be evident to those of skill in the art and, accordingly, the present invention should not be considered in any way limited to the particular plant transformation methods exemplified above.

As hereinbefore described, the nucleotide sequence of interest may be any nucleotide sequence, and may include, for example, positive selectable marker, a knock out cassette, a knock in cassette, a replacement allele and the like.

In preferred embodiments, however, the nucleotide sequence of interest includes a knock out cassette or a knock in cassette as described herein.

The method of the second aspect of the invention is applicable to any cell type. However, in a preferred embodiment, the method of the second aspect of the invention is particularly applicable to plant cells. More preferably, the plant cell is derived from a monocotyledonous plant, and most preferably a cereal crop plant.

In a third aspect, the present invention further extends to a genetically modified cell comprising the nucleic acid construct according to the first aspect of the invention, or a genomically integrated form of the construct.

As referred to herein, a “genomically integrated form” of genetic element (I) comprises any form or part of genetic element (I), which is inserted into the genomic DNA of a cell. As such, the genomically integrated form of genetic element (I) may be all of genetic element (I) or may be a genomically integrated part thereof. As described herein, genetic element (I) is adapted to insert a nucleotide sequence of interest, which is flanked by regions of homology, into a target nucleic acid via homologous recombination. As such, in one preferred embodiment, the genomically integrated form of genetic element (I) may be a nucleotide sequence of interest that has been inserted into the genomic DNA of a cell from genetic element (I) via homologous recombination.

As used herein, the term “genomic DNA” should be understood in its broadest context to include any and all DNA that makes up the genetic complement of a cell. As such, the genomic DNA of a cell should be understood to include chromosomes, mitochondrial DNA, plastid DNA, chloroplast DNA, endogenous plasmid DNA and the like. As such, the present invention contemplates a cell comprising genetic element (I) or a chromosomally integrated form thereof, a mitochondrial DNA integrated form thereof, a plastid DNA integrated form thereof, a chloroplast DNA integrated form thereof, an endogenous plasmid integrated form thereof, and the like.

The cells contemplated by this aspect of the invention include the cells into which the construct is intended to be introduced as well as any other cell or cells which incorporate the construct, which may or may not be involved in the generation of the construct and/or delivering the construct to the target cell.

In one preferred embodiment, the cell is a plant cell. In a more preferred embodiment, the plant cell is a dicotyledonous plant cell, for example, an Arabidopsis sp. cell. In another more preferred embodiment the cell is a monocotyledonous plant cell and more preferably a cereal crop plant cell.

In another preferred embodiment, the cell may also comprise a prokaryotic cell. For example the prokaryotic cell may include an Agrobacterium sp. cell (or other plant transformation competent cell) which carries the nucleic acid construct and which may, for example, be used to transform a plant. In another embodiment, the prokaryotic cell may include an E. coli cell, which may, for example, be used in the construction or cloning of a nucleic acid construct.

In one preferred embodiment, the genetically modified cell of the third aspect of the invention is produced according to the method of the second aspect of the invention.

In a fourth aspect, the present invention should be understood to extend to a multicellular structure comprising one or more cells referred to in the third aspect of the invention.

As referred to herein, a “multicellular structure” includes any aggregation of one or more cells. As such, a multicellular structure specifically encompasses tissues, organs, whole organisms and parts thereof. Furthermore, a multicellular structure should also be understood to encompass multicellular aggregations of cultured cells such as colonies, plant calli, suspension cultures and the like.

As mentioned above, in one preferred embodiment of the invention, the cell is a plant cell and as such, the present invention includes a whole plant, plant tissue, plant organ, plant part, plant reproductive material or cultured plant tissue, comprising one or more plant cells according to the third aspect of the invention.

In a fifth aspect, the present invention provides a method for producing a genetic construct comprising genetic element (I), the method comprising:

(i) providing one or more nucleic acid molecules which together comprise genetic elements (II), (III), (IV) and (V) as shown below:

[NSM1-RSa-RSd-NSM2] (II)

[RSa′-5′RH-RSb] (III)

[RSb′-nsoi-RSc′] (IV)

[RSc-3′RH-RSd′] (V)

wherein:

NSM1 comprises a nucleotide sequence encoding a negative selectable marker, or is optionally absent if a nucleotide sequence encoding a negative selectable marker is present at NSM2;

NSM2 comprises a nucleotide sequence encoding a negative selectable marker, or is optionally absent if a nucleotide sequence encoding a negative selectable marker is present at NSM1;

5′HR comprises a nucleotide sequence which is homologous to a nucleotide sequence 5′ to, and/or inclusive of, a nucleotide sequence in the target nucleic acid molecule;

nsoi comprises a nucleotide sequence of interest to be inserted into the target nucleotide sequence;

3′HR comprises a nucleotide sequence which is homologous to a nucleotide sequence 3′ to, and/or inclusive of, a nucleotide sequence in the target nucleic acid molecule; and

RSa is a nucleotide sequence defining a recombination site which can recombine with RSa′ when acted on by a recombinase; RSb is a nucleotide sequence defining a recombination site which can recombine with RSb′ when acted on by a recombinase, RSc is a nucleotide sequence defining a recombination site which can recombine with RSc′ when acted on by a recombinase and RSd is a nucleotide sequence defining a recombination site which can recombine with RSd′ when acted on by a recombinase; and

(ii) administering one or more recombinases to the one or more nucleic acid molecules comprising genetic elements (II), (III), (IV) and (V) such that RSa and RSa′ recombine to yield the recombination site RSα, RSb and RSb′ recombine to yield the recombination site RSβ, RSc and RSc′ recombine to yield the recombination site RSγ and RSd and RSd′ recombine to yield the recombination site RSδ, thereby generating a nucleic acid construct comprising genetic element (I).

In the method of the fifth aspect of the invention, genetic elements (II), (III), (IV) and (V) may be supplied on one or more nucleic acid molecules. That is, each of the genetic elements may be supplied on a separate nucleic acid molecule or two, three or four of the genetic elements may be provided together on a single nucleic acid molecule. However, in a preferred embodiment, each of genetic elements (II), (III), (IV) and (V) is provided on a separate nucleic acid molecule.

The method of the fifth aspect of the invention allows one or many constructs comprising genetic element (I) to be rapidly and efficiently produced. For example, regions of homology to a target gene may be incorporated into genetic elements (III) and (V), using standard techniques in the art. These elements may then be recombined with genetic elements (II) and (IV) which incorporate the negative selectable markers and nucleotide sequence of interest (e.g., a positive selectable marker), respectively, to generate a construct comprising genetic element (I).

Furthermore, the present invention provides a modular system for producing an array of constructs comprising genetic element (I). For example, in order to produce a range of constructs for different nucleic acid targets, it is only necessary to generate new genetic elements (III) and (V), each having regions of homology to a particular nucleic acid target. However, the same genetic elements (II) and (IV) may be used no matter what 5′RH and 3′RH sequences are incorporated into genetic elements (III) and (V), as the recombination sites in the genetic elements (II), (III), (IV) and (V) remain the same.

Furthermore, a series of constructs comprising genetic element (I), but with different negative selectable markers may be produced by using series of vectors comprising genetic element (II), each carrying different negative selectable markers. Similarly, incorporating different nucleotide sequences of interest into a series of vectors comprising genetic element (IV) would allow the production of a range of constructs comprising genetic element (I), each with a different nucleotide sequence of interest.

In this way, an array of different constructs each comprising different embodiments of genetic element (I), each with different target specificity, different negative selectable markers and different nucleotide sequences of interest can be rapidly and efficiently produced. This may be achieved by selecting appropriate vectors comprising genetic elements (II), (III), (IV) and (V), which have the desired features, and recombining them via the recombination sites.

Preferably, NSM1, NSM2, nsoi, 5′RH and 3′RH are as hereinbefore described with respect to the earlier aspects of the invention.

In a particularly preferred embodiment, RSa, RSa′, RSb, RSb′, RSc, RSc′, RSd and RSd′ comprise lambda phage att sites as hereinbefore defined. In a more preferred embodiment, RSa, RSb, RSc and RSd comprise attR sites and RSa′, RSb′, RSc′ and RSd′ comprise attL sites. In this embodiment, the recombination of the attR sites at RSa, RSb, RSc and RSd and the attL sites at RSa′, RSb′, RSc′ and RSd′ would generate an attB site at each of RSα, RSβ, RSγ and RSδ in genetic element (I).

In an alternate preferred embodiment, RSa, RSb, RSc and RSd comprise attL sites and RSa′, RSb′, RSc′ and RSd′ comprise attR sites. In this embodiment, the recombination of the attL sites at RSa, RSb, RSc and RSd and the attR sites at RSa′, RSb′, RSc′ and RSd′ would generate an attP site at each of RSα, RSβ, RSγ and RSδ in genetic element (I).

As set out above, the reaction of at attL with an attR recombination site is catalysed by bacteriophage lambda integrase (Int), bacteriophage lambda excisionase (Xis) and the E. Coli Integration Host Factor (IHF). Accordingly, when RSa, RSb, RSc and RSd comprise attR sites and RSa′, RSb′, RSc′ and RSd′ comprise attL site or RSa, RSb, RSc and RSd comprise attL sites and RSa′, RSb′, RSc′ and RSd′ comprise attR sites, the recombinase comprises a mixture of bacteriophage lambda integrase (Int), bacteriophage lambda excisionase (Xis) and the E. Coli Integration Host Factor (IHF).

In another embodiment, RSa, RSb, RSc and RSd comprise attP sites and RSa′, RSb′, RSc′ and RSd′ comprise attB sites. In yet another embodiment RSa, RSb, RSc and RSd comprise attB sites and RSa′, RSb′, RSc′ and RSd′ comprise attP sites. In these embodiments, the recombination of the attB and attP sites would generate an attL or an attR site at each of RSα, RSβ, RSγ and RSδ in genetic element (I). As set out above, the reaction of at attB with an attP recombination site is catalysed by bacteriophage lambda integrase (Int) and the E. Coli Integration Host Factor (IHF). Accordingly, when RSa, RSb, RSc and RSd comprise attP sites and RSa′, RSb′, RSc′ and RSd′ comprise attB site or RSa, RSb, RSc and RSd comprise attB sites and RSa′, RSb′, RSc′ and RSd′ comprise attP sites, the recombinase comprises a mixture of bacteriophage lambda integrase (Int) and the E. Coli Integration Host Factor (IHF).

However, as set out above, in a preferred embodiment, RSα, RSβ, RSγ and RSδ in genetic element (I) comprise attB recombination sites. Therefore, in a particularly preferred embodiment of the sixth aspect of the invention, RSa, RSb, RSc and RSd comprise attR sites and RSa′, RSb′, RSc′ and RSd′ comprise attL sites which, after recombination, would generate an attB site at RSα, RSβ, RSγ and RSδ in genetic element (I).

In a sixth aspect, the present invention contemplates a genetic construct comprising genetic element (II) as defined in the sixth aspect of the invention.

In a preferred embodiment, NSM1 and NSM2 in the construct of the sixth aspect of the invention are as hereinbefore described with reference to the first aspect of the invention.

In another preferred embodiment, RSa and RSd in the construct of the sixth aspect of the invention are as hereinbefore described with regard to the fifth aspect of the invention.

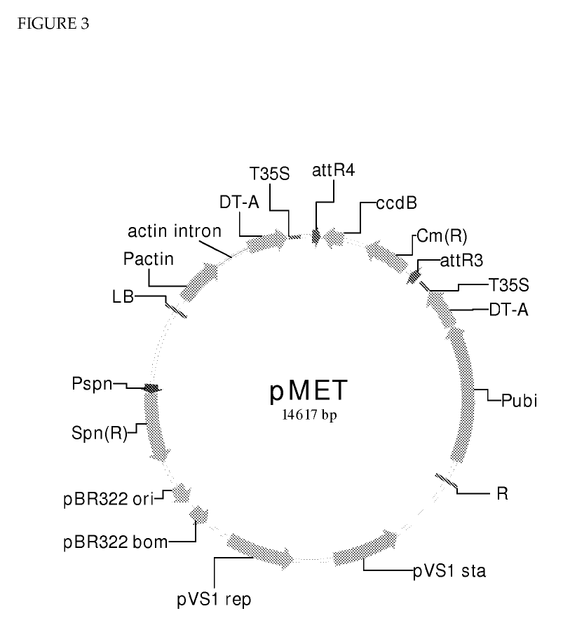

In a particularly preferred embodiment, the construct of the sixth aspect of the invention comprises a pMET vector as shown in FIG. 3.

Referring to FIG. 3, pMET is a pCAMBIA binary vector backbone where 2 DT-A gene cassettes (promoter::gene::terminator) are cloned in between the T-DNA right and left border element for Agrobacterium transformation. In the illustrated embodiment, the promoters used for DT-A expression are the rice actin promoter (Pact) and the ubiquitin promoter (Pubi) from maize. Both cassettes are ended with the 35S terminator. In between the 2 DT-A cassettes is cloned the attR4-attR3 recombination cassette from pDEST R4-R3 (Invitrogen).

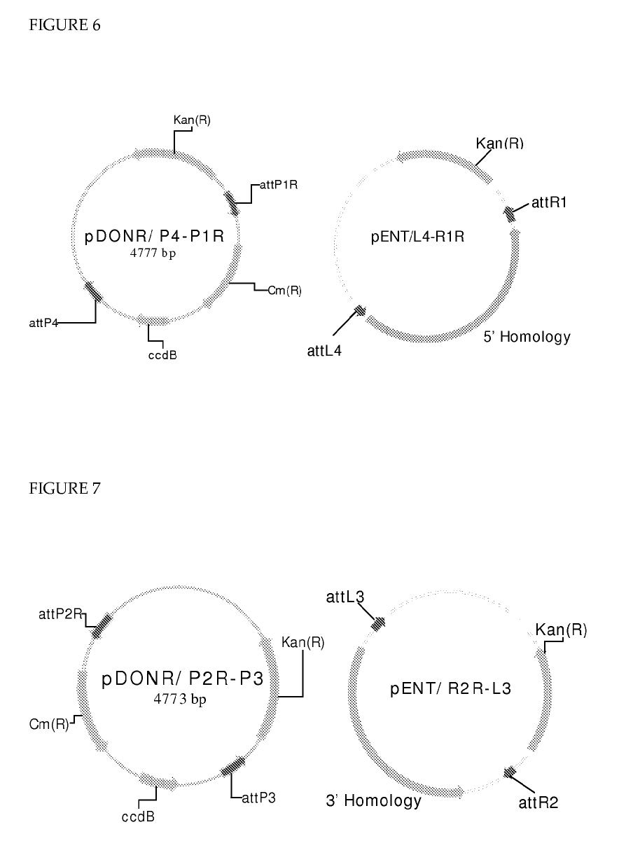

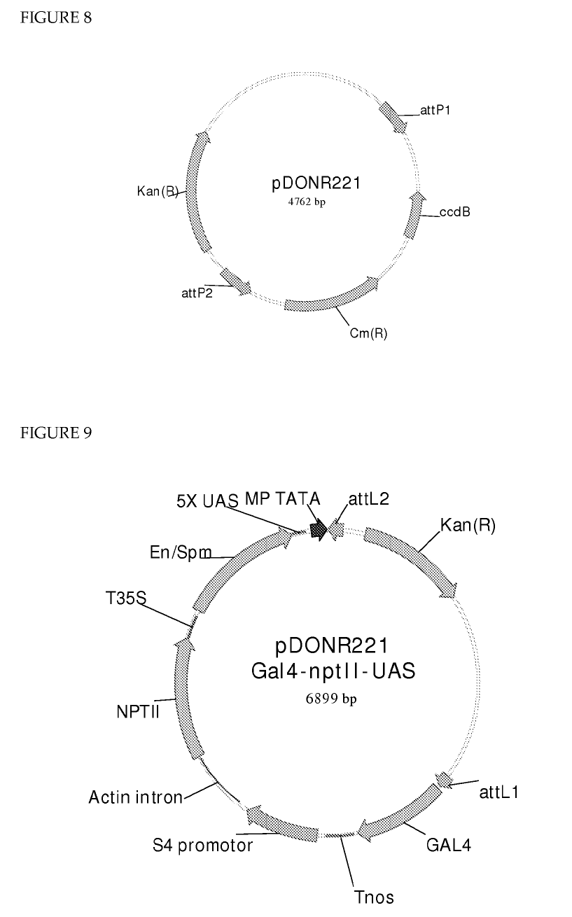

pMET is adapted to work in association with 3 entry vectors which are derived from pDONR P4-P1R (FIG. 6), pDONR P2R-P3 (FIG. 7) and pDONR 221 (FIG. 8), respectively.

5′ homology and 3′ homology regions, which promote homologous recombination of the final construct with a target nucleic acid may cloned into pDONR P4-P1R and pDONR P2R-P3 to generate constructs comprising genetic elements (III) and (V) as hereinbefore defined.

In a preferred embodiment, the 5′ homology region is inserted into pDONR P4-P1R vector via a BP-clonase mediated reaction to generate construct the “entry” vector pENT/L4-R1R (FIG. 6) comprising the 5′ region of homology flanked by attL4 and attR1 sites.

In another preferred embodiment, the 3′ homology region is inserted into pDONR P2R-P3 vector via a BP-clonase mediated reaction to generate construct the “entry” vector pENT/R2R-L3 (FIG. 7) comprising the 3′ region of homology flanked by an attL3 and attR2 sites.

A multiple cloning site (such as that from pCAMBIA0380) may be cloned into pDONR221 and a nucleotide sequence of interest may be cloned into this multiple cloning site. Alternatively, a nucleotide sequence of interest may be directly gateway-cloned as well in between the att sites in pDONR221. Once the nucleotide sequence of interest has been cloned into pDONR221, this vector carries an embodiment of genetic element (IV), as previously defined.

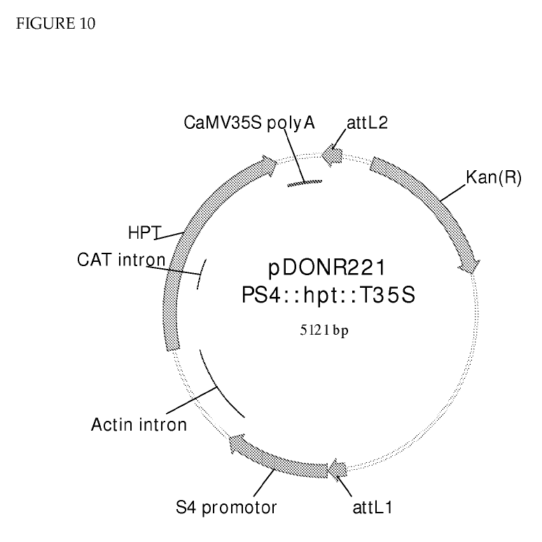

As hereinbefore described, several pDONR221 vectors, each comprising a different nucleotide sequence of interest between the att sites, may be produced. Exemplary pDONR221-derived entry vectors are shown in FIGS. 9 and 10. FIG. 9 shows a pDONR221-derived entry vector carrying a “knock in” cassette (as described with reference to pMET KI), while FIG. 10 shows a pDONR-derived entry vector carrying a “knock out” cassette (as described with reference to pMET KO.

In the illustrated embodiment, in order to produce a targeting vector, the pMET vector (carrying the negative selectable markers) and an appropriate pDONR221-derived entry vector (such as pDONR221/Gal4::nptII::UAS or pDONR221/PS4::hpt::T35S) carrying the nucleotide sequence of interest, are selected. Regions of homology to the target gene are then cloned into the pDONR P4-P1R- and pDONR P2R-P3-derived entry vectors to generate the entry vectors pENT/L4-R1R and pENT/R2R-L3. The pMET vector and the entry vectors are then assembled via recombination through the att recombination sites. Again, in the illustrated embodiment, correct assembly of the vector is promoted by the preferential recombination of attL1 with attR1, attL2 with attR2 and attL3 with attR3.

The present invention also contemplates a genetic construct of the sixth aspect of the invention, as described herein, when used according to the method of the fifth aspect of the invention.

In a seventh aspect, the present invention also provides a kit for performing the method of the fifth aspect of the invention, wherein the kit comprises at least the genetic construct of the sixth aspect of the invention.

The kit of the seventh aspect of the invention may further comprise other components such as instructions for use; nucleic acid molecules comprising genetic elements (III), (IV) and/or (V); enzymes and reagents for performing the method of the fifth aspect of the invention; and the like.

The present invention is further described by the following non-limiting examples:

### Example 1

### Construction of a Targeted “Knock in” Vector, pMET KI

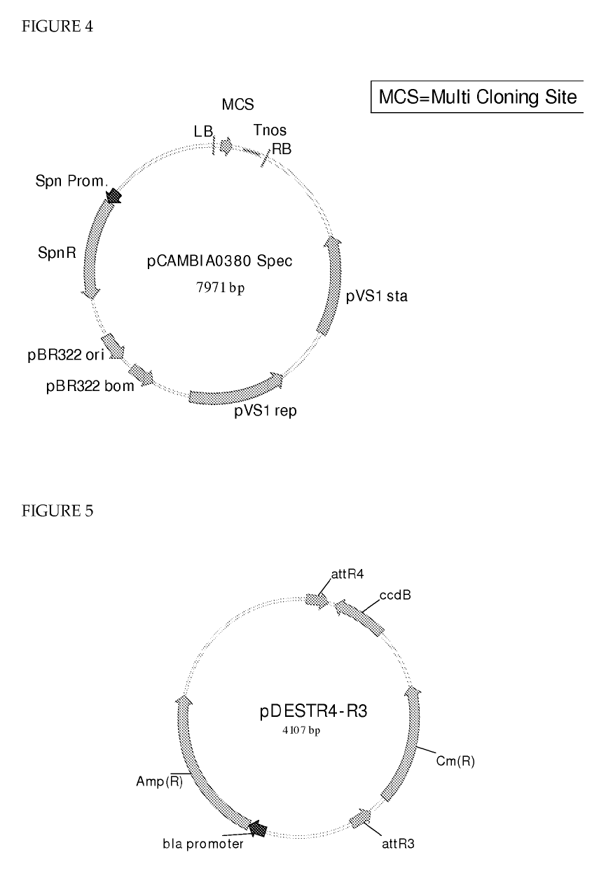

A DT-A gene operably connected to a rice actin promoter (Pact) followed by its first intron was cloned in between the AvrII-AscI sites of a modified pCAMBIA0380 vector (FIG. 4), upstream of the CaMV 35S terminator/poly A sequence, to form a Pact::DT-A gene::terminator 35S expression cassette.

The DT-A fragment used codes for an amino-acid sequence comprising amino acid residues 35 to 226 of the P00588 protein (NCBI accession number), with two modifications. First, 3 amino-acid residues have been added before residue 35. The first of these Methionine (ATG, translation start), the second is aspartic acid while the third is proline. The second modification made to the protein comprises the addition of a stop codon after amino acid residue 226.

The attR4-attR3 gateway-recombination cassette from the Invitrogen pDESTR4-R3 plasmid (FIG. 5) was then PCR-amplified and cloned between the HindIII-SpeI sites of the newly constructed pCAMBIA0380-DT-A binary vector. At the same time, a ubiquitin promoter::DT-A gene::terminator 35S expression cassette was PCR-amplified and cloned into this vector in the PvuI-SpeI sites to form the pMET binary vector comprising two DT-A expression cassettes which are directed towards each other in between the left and right T-DNA borders of the modified pCAMBIA0380 binary vector.

To form the pMET KI gene targeting vector, pMET was placed in contact with modified pENT/L4-R1R (FIG. 6) and pENT/R2R-L3 (FIG. 7) plasmids wherein the 5′ region of homology comprises a nucleotide sequence which hybridises to a promoter region and the 3′ region of homology comprises a nucleotide sequence which hybridises to the gene which is operably connected to the promoter region. Once incorporated into the targeting vector, these regions of homology serve to direct the insertion of the knock in cassette between the in between the targeted promoter and gene. pDONR221/Gal4::nptII::UAS (FIG. 9) was used as the final entry vector. This vector comprises the Gal4::nptII::UAS knock in cassette described previously.

An LR gateway-recombination reaction was then used (according to the manufacturer's instructions) to combine all of the fragments in the correct order to generate the pMET KI targeting vector.

Standard protocols were followed for the gateway-recombination reactions. However, for the final LR gateway-recombination reaction, an extended incubation time of 24 hours or more (instead of 16 hours as recommended) was used, which allows the recovery of more recombinant clones. Furthermore, because the final LR gateway-recombination reaction is a multi-site assembly, the “LR Clonase Plus Enzyme mix” was used instead of standard LR Clonase Enzyme mix.

### Example 2

### Construction of a Targeted “Knock Out” Vector, pMET KO

A DT-A gene operably connected to a rice actin promoter (Pact) followed by its first intron was cloned in between the AvrII-AscI sites of a modified pCAMBIA0380 vector (FIG. 4), upstream of the CaMV 35S terminator/poly A sequence, to form a Pact::DT-A gene::terminator 35S expression cassette.

The DT-A fragment used codes for an amino-acid sequence comprising amino acid residues 35 to 226 of the P00588 protein (NCBI accession number), with two modifications. First, 3 amino-acid residues have been added before residue 35. The first of these Methionine (ATG, translation start), the second is aspartic acid while the third is proline. The second modification made to the protein comprises the addition of a stop codon after amino acid residue 226.

The attR4-attR3 gateway-recombination cassette from the Invitrogen pDESTR4-R3 plasmid (FIG. 5) was then PCR-amplified and cloned between the HindIII-SpeI sites of the newly constructed pCAMBIA0380-DT-A binary vector. At the same time, a ubiquitin promoter::DT-A gene::terminator 35S expression cassette was PCR-amplified and cloned into this vector in the PvuI-SpeI sites to form the pMET binary vector comprising two DT-A expression cassettes which are directed towards each other in between the left and right T-DNA borders of the modified pCAMBIA0380 binary vector.

To form the pMET KO targeting vector, pMET is placed in contact with modified pENT/L4-R1R (FIG. 6) and pENT/R2R-L3 (FIG. 7) plasmids wherein the 5′ and 3′ regions of homology comprises a nucleotide sequences which are homologous to the coding region of the targeted gene. Once incorporated into the targeting vector, these regions of homology serve to direct the insertion of the knock out cassette into the coding region of the targeted gene. pDONR221/PS4::hpt::T35S (FIG. 10) is used as the final entry vector. This vector comprises the PS4::hpt::T35S knock out cassette as described previously.

An LR gateway-recombination reaction then combines all of the fragments in the correct order to generate the pMET KO gene-targeting vector.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Also, it must be noted that, as used herein, the singular forms “a”, “an” and “the” include plural aspects unless the context already dictates otherwise. Thus, for example, reference to “a nucleotide sequence of interest” includes a single nucleotide sequence as well as two or more nucleotide sequences; “a plant cell” includes a single cell as well as two or more cells; and so forth.

1-45. (canceled)

46. A nucleic acid construct for inserting a nucleotide sequence of interest into a target nucleic acid molecule via homologous recombination, the nucleic acid construct comprising genetic element (I) as shown below:
[NSM1-RSα-5′HR-RSβ-nsoi-RSγ-3′HR-RSδ-NSM2] (I)
wherein:
NSM1 comprises a nucleotide sequence encoding a negative selectable marker, or optionally may be absent if a nucleotide sequence encoding a negative selectable marker is present at NSM2;
RSα comprises a nucleotide sequence defining a first recombination site;
5′HR comprises a nucleotide sequence which is homologous to a nucleotide sequence 5′ to, and/or inclusive of, a nucleotide sequence in the target nucleic acid molecule;
RSβ comprises a nucleotide sequence defining a second recombination site;
nsoi comprises a nucleotide sequence of interest to be inserted into the target nucleic acid molecule;
RSγ comprises a nucleotide sequence defining a third recombination site;
3′HR comprises a nucleotide sequence which is homologous to a nucleotide sequence 3′ to, and/or inclusive of, a nucleotide sequence in the target nucleic acid molecule;
RSδ comprises a nucleotide sequence defining a fourth recombination site;
NSM2 comprises a nucleotide sequence encoding a negative selectable marker, or optionally may be absent if a nucleotide sequence encoding a negative selectable marker is present at NSM1;
wherein homologous recombination between the regions of homology, 5′HR and/or 3′HR, and homologous nucleotide sequences in the target nucleic acid molecule results in insertion of the portion of genetic element (I) which is bounded by, and optionally inclusive of, 5′HR and 3′HR, into the target nucleic acid molecule.

47. The targeting construct of claim 46 wherein nsoi comprises a nucleotide sequence encoding a positive selectable marker.

48. The targeting construct of claim 46 or 47 wherein each of RSα, RSβ, RSγ and RSδ comprise lambda phage att sites.

49. The genetic construct of claim 46 wherein the genetic construct further comprises T-DNA border sequences flanking genetic element (I).

50. A method for inserting a nucleotide sequence of interest into a nucleic acid molecule in a cell via homologous recombination, the method comprising administering to the cell the nucleic acid construct of claim 46; and selecting transformants which comprise the inserted nucleotide sequence of interest on the basis of the presence and/or expression of the nucleotide sequence of interest in the cell, and/or the absence of expression of one or both of the negative selectable markers NSM1 and NSM2 in the cell.

51. The method of claim 50 wherein the cell is a plant cell.

52. A genetically modified cell comprising either the construct of claim 46 or a genomically integrated form of said construct.

53. The genetically modified cell of claim 52 wherein the cell is a plant cell.

54. The genetically modified cell of claim 52 wherein the cell is produced according to the method of claim 50.

55. A multicellular structure comprising one or more of the genetically modified cells of claim 52.

56. The multicellular structure of claim 55 wherein the multicellular structure is a plant.

57. A method for producing a genetic construct comprising genetic element (I) as defined in claim 46, the method comprising:
providing one or more nucleic acid molecules which together comprise genetic elements (II), (III), (IV) and (V) as shown below:
[NSM1-RSa-RSd-NSM2] (II)
[RSa′-5′RH-RSb] (III)
[RSb′-nsoi-RSc′] (IV)
[RSc-3′RH-RSd′] (V)
wherein:
NSM1 comprises a nucleotide sequence encoding a negative selectable marker, or optionally may be absent if a nucleotide sequence encoding a negative selectable marker is present at NSM2;
NSM2 comprises a nucleotide sequence encoding a negative selectable marker, or optionally may be absent if a nucleotide sequence encoding a negative selectable marker is present at NSM1;
5′HR comprises a nucleotide sequence which is homologous to a nucleotide sequence 5′ to, and/or inclusive of, a nucleotide sequence in the target nucleic acid molecule;
nsoi comprises a nucleotide sequence of interest to be inserted into the target nucleic acid molecule;
3′HR comprises a nucleotide sequence which is homologous to a nucleotide sequence 3′ to, and/or inclusive of, a nucleotide sequence in the target nucleic acid molecule; and
RSa is a nucleotide sequence defining a recombination site which can recombine with RSa′ when acted on by a recombinase; RSb is a nucleotide sequence defining a recombination site which can recombine with RSb′ when acted on by a recombinase, RSc is a nucleotide sequence defining a recombination site which can recombine with RSc′ when acted on by a recombinase and RSd is a nucleotide sequence defining a recombination site which can recombine with RSd′ when acted on by a recombinase; and
(ii) administering one or more recombinases to the one or more nucleic acid molecules comprising genetic elements (II), (III), (IV) and (V) such that RSa and RSa′ recombine to yield the recombination site RSα, RSb and RSb′ recombine to yield the recombination site RSβ, RSc and RSc′ recombine to yield the recombination site RSγ, and RSd and RSd′ recombine to yield the recombination site RSδ, thereby generating a nucleic acid construct comprising genetic element (I).

58. The method of claim 57 wherein each of genetic elements (II), (III), (IV) and (V) are provided on separate nucleic acid molecules or vectors.

59. The method of claim 57 wherein the nsoi comprises a nucleotide sequence encoding a positive selectable marker.

60. The method of claim 57, wherein RSa, RSa′, RSb, RSb′, RSc, RSc′, RSd and RSd′ comprise lambda phage att sites.

61. A nucleic acid construct comprising genetic element (II) as defined in claim 57.

62. The nucleic acid construct of claim 61 wherein RSa and RSd comprise lambda phage att sites.

63. A kit for performing the method of claim 57, the kit comprising the genetic construct of claim 61 together with instructions for performing the method of claim 57.
